(19) United States
(12) Patent Application Publication
(10) Pub. No.: US 2025/0208040 A1
(43) Pub. Date: Jun. 26, 2025

(54) PREPARATION METHOD OF NANOGEL FLUORESCENT SENSOR

(71) Applicant: Lishui University, Lishui (CN)

(72) Inventors: Xiaoli Sun, Lishui (CN); Luoxing Yang, Lishui (CN); Yueyi Xia, Lishui (CN); Muhua Wang, Lishui (CN)

(21) Appl. No.: 18/904,092

(22) Filed: Oct. 2, 2024

(30) Foreign Application Priority Data
Dec. 22, 2023 (CN) 202311779719.5

(51) Int. Cl. G01N 21/64; G01N 33/14

(52) U.S. Cl. CPC G01N 21/6428; G01N 33/146

(57) ABSTRACT

A preparation and use of a green alternative template molecularly imprinted-carbon quantum dot nanogel fluorescent sensor. The morphology, chemical structure, and optical properties of the fluorescent sensor were investigated using techniques such as Transmission Electron Microscopy (TEM), X-ray Photoelectron Spectroscopy (XPS), Fourier Transform Infrared Spectroscopy (FTIR), Fluorescence Spectroscopy. Using Bisphenol A as a detection model, the selectivity and sensitivity of the sensor were evaluated based on changes in fluorescence intensity. The green alternative template molecularly imprinted-carbon quantum dot nanogel sensor provided by the present disclosure exhibits excellent selectivity, high sensitivity, and a low detection limit. It can achieve highly selective recognition of Bisphenol A in plastic barrel-packed Baijiu samples and is suitable for the enrichment and purification of Bisphenol A in organic phases. The prepared green alternative template molecularly imprinted-carbon quantum dot nanogel fluorescent sensor can be used for highly selective fluorescence detection of Bisphenol A substances in complex matrices.

### CROSS REFERENCE TO RELATED APPLICATIONS

The present application claims the benefit of Chinese Patent Application No. 202311779719.5 filed on Dec. 22, 2023, the contents of which are incorporated herein by reference in their entirety.

### TECHNICAL FIELD

The present disclosure relates to the technical field of molecular imprinting, and in particular to a preparation method of a nanogel fluorescent sensor.

### BACKGROUND

Bisphenol A (BPA), is one of the most widely used chemical raw materials in the world and is often utilized as a monomer for synthesizing polymers such as polycarbonates. Additionally, BPA is a common endocrine disruptor, and excessive exposure can harm the endocrine, nervous, and immune systems of both humans and animals, while also increasing the incidence of various cancers, including leukemia and ovarian cancer. Although many countries, including the European Union, the United States, China, and Canada, have enacted specific laws and regulations prohibiting the addition of BPA to baby bottles and other food-related containers, BPA still enters the environment through dust, sewage, and other pathways due to the widespread use of BPA-related products. Therefore, it is crucial to assess the content and distribution of BPA in food and environmental samples. Given the low levels of BPA present in food and environmental samples and the significant matrix interference, developing an adsorbent material with high selectivity and adsorption capacity is of utmost importance.

Carbon quantum dots (CQDs) represent a novel quasi-zero-dimensional nano-fluorescent material that has emerged in the realm of carbon nanomaterials, following the advent of two-dimensional graphene and one-dimensional carbon nanotubes. CQDs exhibit unique optical properties such as high photoluminescence, resistance to photobleaching, and optical stability. They have garnered extensive attention and research from scholars due to their advantages of low cost, good biocompatibility, environmental friendliness, and ease of functionalization. Furthermore, CQDs can be surface-passivated and functionalized with other types of materials to obtain nanostructured composites with excellent performance. In recent years, CQDs have made significant breakthroughs and demonstrated notable advantages in applications such as bioimaging, sensing, and drug delivery.

Molecular Imprinting Technique (MIT) is a technology that mimics antigen-antibody recognition to construct Molecularly Imprinted Polymers (MIPs) with specific recognition sites. MIPs are high-polymer compounds obtained through polymerization reactions among various functional monomers in the presence of a template molecule. After the removal of the template, molecularly imprinted cavities that are complementary to the three-dimensional shape and interaction sites of the template molecule are formed within the MIPs. The prepared MIPs possess three major characteristics: predetermination, recognition, and practicality, enabling selective separation and enrichment of target compounds in complex samples and enhancing detection sensitivity. Currently, MIT has been widely applied in areas such as chromatographic separation, solid-phase extraction, biomimetic sensing, enzyme-mimicking catalysis, and clinical drug analysis.

Utilizing the sol-gel method, highly selective MIPs are combined with highly sensitive CQDs to prepare CQDs@MIPs, where the sol-gel method allows CQDs to be attached within the MIPs material, resulting in molecularly imprinted polymers with fluorescent emission.

Currently, there are two major issues in the preparation of molecularly imprinted polymers: \textcircled{1} The problem of template molecule leakage in imprinted materials. Due to the need for adding a large amount of template molecules during the preparation of imprinted materials, even intensive extraction methods such as prolonged Soxhlet extraction, ultrasonic extraction, or accelerated solvent extraction cannot completely remove the template molecules. The template molecules that remain deep within the imprinted material will gradually leak out during subsequent usage, leading to inaccurate analysis results. \textcircled{2} Insufficient and uneven affinity sites with weak affinity. The preparation process requires the introduction of acidic or basic conditions to promote the hydrolysis of silane reagents. The introduction of these acidic or basic conditions can affect the template-monomer interactions in the non-covalent pre-polymerization solution, resulting in issues such as a limited number of affinity sites, weak affinity, and unevenness in the synthesized molecularly imprinted materials.

### SUMMARY

The objective of the present disclosure is to provide a method for the preparation and use of a green alternative template molecularly imprinted-carbon quantum dot nanogel fluorescent sensor that exhibits high selectivity and rapid enrichment detection for Bisphenol A (BPA). Using BPA as the detection model, the green alternative template molecularly imprinted-carbon quantum dot nanogel fluorescent sensor (Si-CQDs@DMIPs) was prepared by combining semi-covalently synthesized alternative template monomer complexes with CQDs through a sol-gel method. This sensor was then applied for the detection of BPA in Baijiu, achieving rapid enrichment and detection of BPA in Baijiu samples.

In order to achieve the above objects, the present disclosure adopts the following technical solutions:

In the first aspect, the present disclosure provides a preparation method of a nanogel fluorescent sensor, comprising the following steps:
(1) preparing carbon quantum dots:
(1-1) taking anhydrous citric acid as a carbon source, taking 3-aminopropyltriethoxysilane as a dispersant, sealing, and carrying out a reaction;
(1-2) after the reaction is finished, centrifuging is performed to remove unreacted solids, obtaining silane-functionalized quantum dots, dispersing the silane-functionalized quantum dots in anhydrous ethanol, and storing at 4° C. for future use;
(2) preparing an alternative template-monomer covalent complex:
(2-1) dissolving bisphenol A, acting as an alternative for template molecules phenolphthalein and 1,1,1-tris(4-hydroxyphenyl)ethane, in N,N-dimethylformamide, then adding a functional monomer isocyanatopropyltriethoxysilane, and subsequently performing a sealed reaction at 60-90° C. for 24-72 h;
(2-2) after the reaction is finished, removing N,N-dimethylformamide from the system to obtain a bisphenol A alternative template-monomer covalent complex, and storing the bisphenol A alternative template-monomer covalent complex under high-purity nitrogen sealing for later use;
(3) preparing an alternative template molecularly imprinted nanogel fluorescent sensor using carbon quantum dots as a fluorescent unit:
(3-1) dissolving the bisphenol A alternative template-monomer covalent complex in anhydrous ethanol to form a pre-reaction solution;
(3-2) dropwise adding a silane-functionalized quantum dot ethanol solution into the pre-reaction solution under mechanical stirring, dropwise adding tetraethyl orthosilicate and an ammonia solution in sequence, and stirring the mixture at room temperature for 2-4 h;
(3-3) after the reaction is completed, performing solid-liquid separation on the mixture to obtain a gel material; and
(3-4) adding the gel material into a dimethyl sulfoxide aqueous solution, maintaining the mixture at 160-200° C. for 2-4 h to remove the bisphenol A alternative template molecule, separating out a solid, then cleaning the solid, and carrying out vacuum drying to obtain the alternative template molecularly imprinted nanogel fluorescent sensor.

Preferably, in step (1-1), anhydrous citric acid is fully dissolved in 3-aminopropyltriethoxysilane with the assistance of ultrasound, where a molar ratio of citric acid to 3-aminopropyltriethoxysilane is 1:14-16; and/or, performing the reaction in the sealed environment is maintained at 180-240° C. for 3-6 h, where performing the reaction in the sealed environment is maintained at 210+5° C. for 6 h; and performing the reaction in the sealed environment is carried out in either a graphene reaction kettle or a Teflon-lined autoclave.

Preferably, in step (1-2), the silane-functionalized quantum dots are dispersed in anhydrous ethanol according to a volume ratio of 1:1.

Preferably, in step (2-1), a molar ratio of the bisphenol A alternative template molecule to the functional monomer isocyanatopropyltriethoxysilane is 1:1.5-4, where the molar ratio of the bisphenol A alternative template molecule to the functional monomer isocyanatopropyltriethoxysilane is 1:2-3; and/or, the sealed reaction is conducted in a high-pressure glass reaction tube, where the sealed reaction is conducted at 80±5° C. for 48-50 h; and/or, N,N-dimethylformamide is a super-dry solvent.

Preferably, in step (3-1), the bisphenol A alternative template-monomer covalent complex is dissolved in anhydrous ethanol by ultrasound, where a concentration of the bisphenol A alternative template-monomer covalent complex is 0.01-0.1 mol/L.

Preferably, in step (3-2), a mass ratio of the bisphenol A alternative template-monomer covalent complex to the silane-functionalized quantum dots in the pre-reaction solution is 1:5-20, where the mass ratio of the bisphenol A alternative template-monomer covalent complex to the silane-functionalized quantum dots in the pre-reaction solution is 1:10; and/or, a mechanical stirring speed is 200-400 rpm; and/or, a molar ratio of tetraethyl orthosilicate to the bisphenol A alternative template-monomer covalent complex is 7-10:1, where the molar ratio of tetraethyl orthosilicate to the bisphenol A alternative template-monomer covalent complex is 8:1; and/or, a ratio of the ammonia solution to the bisphenol A alternative template-monomer covalent complex is 1-2:1 mL/mmol, where the ratio of the ammonia solution to the bisphenol A alternative template-monomer covalent complex is 1:1 mL/mmol.

Preferably, in step (3-3), the solid-liquid separation method is centrifugation, where control parameters of the centrifugation are: a rotation speed is 8000-12000 rpm, and a centrifugation time is 10-15 min.

Preferably, in step (3-4), a volume ratio of dimethyl sulfoxide to water is 5-10:1.

In the second aspect, the present disclosure provides a bisphenol A detection kit, comprising the nanogel fluorescent sensor obtained by the preparation method described in the first aspect.

In the third aspect, the present disclosure provides a method for detecting bisphenol A in plastic barrel-packed Baijiu, using the kit described in the second aspect, and the method comprises the following steps:
mixing a nanogel fluorescent sensor in the kit with Baijiu, and measuring a fluorescence change rate in a system; and
calculating a content of bisphenol A in Baijiu based on the fluorescence change rate.

Preferably, a final concentration of the nanogel fluorescent sensor in the system is 0.1-0.5 mg/L.

The advantages of the present disclosure compared with the prior art are as follows: \textcircled{1} Phenolphthalein (PP) or 1,1,1-tris(4-hydroxyphenyl)ethane is employed as a semi-covalent imprinting template for bisphenol A, which not only prevents template leakage but also significantly enhances imprinting selectivity. \textcircled{2} The sol-gel method is utilized to combine the high selectivity of the imprinting material with the high sensitivity of carbon quantum dots (CQDs). Additionally, CQDs are attached to the inner portion of the molecularly imprinted polymers (MIPs) material framework, resulting in a fluorescent molecularly imprinted polymer. \textcircled{3} During the synthesis process, the template and monomers are covalently bound, making them resistant to the influence of acids and bases. However, during the recognition process, they bind through non-covalent interactions, leading to rapid recognition speed and uniform site affinity. This addresses issues such as a limited number of affinity sites, weak and inhomogeneous affinity in the synthesis of molecularly imprinted materials. \textcircled{4} The prepared double green alternative template molecularly imprinted-carbon quantum dot nanogel fluorescent sensor can be used for highly selective detection of bisphenol A in complex matrices like Baijiu, with no risk of template leakage and reliable results.

### BRIEF DESCRIPTION OF THE DRAWINGS

FIG. 1 shows a scanning electron microscope (SEM) image of a green alternative template molecularly imprinted-carbon quantum dots nanogel fluorescent sensor (Si-CQDs@DMIPs) in Embodiment 1 of the present disclosure at different magnifications, where image A is at 200 nm, and image B is at 50 nm;

FIG. 2 shows an X-ray photoelectron spectroscopy (XPS) spectrum of silane-functionalized quantum dots in Embodiment 1 of the present disclosure;

FIG. 3 shows a fluorescence spectrum of Si-CQDs under different excitation wavelengths in Embodiment 1 of the present disclosure;

FIG. 4 shows a fluorescence spectrum of Si-CQDs@DMIPs under different excitation wavelengths in Embodiment 1 of the present disclosure;

FIG. 5 shows a Stern-Volmer plot for the determination of bisphenol A (BPA) at different concentrations using Si-CQDs@DMIPs in Embodiment 1 of the present disclosure;

FIG. 6 shows a Stern-Volmer plot for the determination of bisphenol A (BPA) at different concentrations using Si-CQDs@NIPs in Embodiment 1 of the present disclosure;

FIG. 7 shows a selectivity evaluation experiment of Si-CQDs@DMIPs in Embodiment 1 of the present disclosure;

FIG. 8 shows a linear range graph for the detection of BPA using Si-CQDs@DMIPs in Embodiment 1 of the present disclosure;

FIG. 9 shows a linear range graph for the detection of BPA using Si-CQDs@NIPs in Embodiment 1 of the present disclosure; and

FIG. 10 shows a schematic diagram of the preparation of Si-CQDs@DMIPs in Embodiment 1 of the present disclosure.

### DETAILED DESCRIPTION OF THE EMBODIMENTS

In the description of the embodiments of the present disclosure, the term “ultra-dry solvent” refers to a solvent with a water content below 5 ppm.

In the description of the embodiments of the present disclosure, the ammonia solution used is commercially available with a mass concentration of 28%.

The technical solution of the present disclosure is further described in detail below with reference to specific embodiments:

### Embodiment 1

This embodiment provides a method for preparing a nanogel fluorescent sensor (Si-CQDs@DMIPs), with the principle illustrated in FIG. 10: Firstly, Si-CQDs are prepared through a simple one-pot hydrothermal method. Under high temperature conditions, during the synthesis of Si-CQDs, anhydrous citric acid undergoes carbonization at high temperatures, and the amino groups on APTES react with the carboxyl groups of anhydrous citric acid through an acylation reaction, resulting in photoluminescent silane-functionalized CQDs. With the catalysis of ammonia solution, PP-ICPTES facilitates the polymerization reaction between ICPTES and TEOS on the surface of Si-CQDs to form a gel coating. This coating effectively embeds the Si-CQDs into the binding sites, thereby promoting the successful preparation of Si-CQDs@DMIPs. The template molecules are removed by solvent reflux. Subsequently, Si-CQDs@DMIPs with specific recognition ability for bisphenol A are obtained. The specific preparation method is as follows:

### (1) Preparation and Structural Characterization of Nanogel Fluorescent Sensor (Si-CQDs@DMIPs):

Preparation of silane-functionalized quantum dots (silane-functionalized CQDs): Using 5 g of citric acid as the carbon source, it was fully dissolved in 30 mL of 3-aminopropyltriethoxysilane (APTES) with the assistance of ultrasonication. Subsequently, the mixture was added to a Teflon-lined autoclave and reacted at 210° C. for 6 h. After the reaction was finished, the unreacted solids were removed by centrifugation, and the obtained silane-functionalized quantum dots were dispersed in anhydrous ethanol at a volume ratio of 1:1. The dispersion was stored at 4° C. for later use.

Preparation of bisphenol A alternative template-monomer complex: 3.1832 g (10 mmol) of phenolphthalein (PP) was dissolved in 15 mL of super-dry DMF solvent with stirring at room temperature. After complete dissolution, 4.8 mL (20 mmol) of isocyanatopropyltriethoxysilane (ICPTES) was added. The mixture was then sealed in a glass pressure-resistant reaction tube and reacted at 80° C. for 48 h. Upon completion of the reaction, DMF was removed by vacuum distillation at 80° C. The obtained transparent viscous substance was the alternative template-monomer covalent complex, either PP-ICPTES or THPE-ICPTES, which was sealed and stored under nitrogen gas.

Preparation of Si-CQDs@DMIPs: 1 mmol (0.739 g) of the bisphenol A alternative template-monomer complex was dissolved in 20 mL of anhydrous ethanol to form a pre-reaction solution. Under mechanical stirring, a solution of silane-functionalized CQDs was added dropwise. The amount of silane-functionalized CQDs solution added was based on a mass ratio of 1:10 between the bisphenol A alternative template-monomer covalent complex and the silane-functionalized quantum dots. Subsequently, 8 mmol of TEOS and 1 mL of ammonia solution were added dropwise in sequence. The mixture was stirred at room temperature for 3 h. After the completion of the reaction, the mixture was transferred to a glass centrifuge tube and centrifuged at 10,000 rpm for 10 min. The dried Si-CQDs@DMIPs sample was then placed in a dimethyl sulfoxide: water (v/v=5:1) solution and heated at approximately 180° C. for 3 h to remove the alternative template molecule PP. Finally, the Si-CQDs@DMIPs were placed in a vacuum drying oven and dried for 6 h. The obtained sample was stored in a sample bottle at room temperature.

Preparation of the non-imprinted material (Si-CQDs@NIPs) for comparison: The preparation of the non-imprinted material is similar to the method for the imprinted material, with the difference being that no alternative template-monomer covalent complex is added to the pre-reaction solution when preparing the non-imprinted material. Instead, 3 mmol of 3-aminopropyltriethoxysilane (APTES)-ethanol solution is used. All other operations are the same as those for the imprinted material.

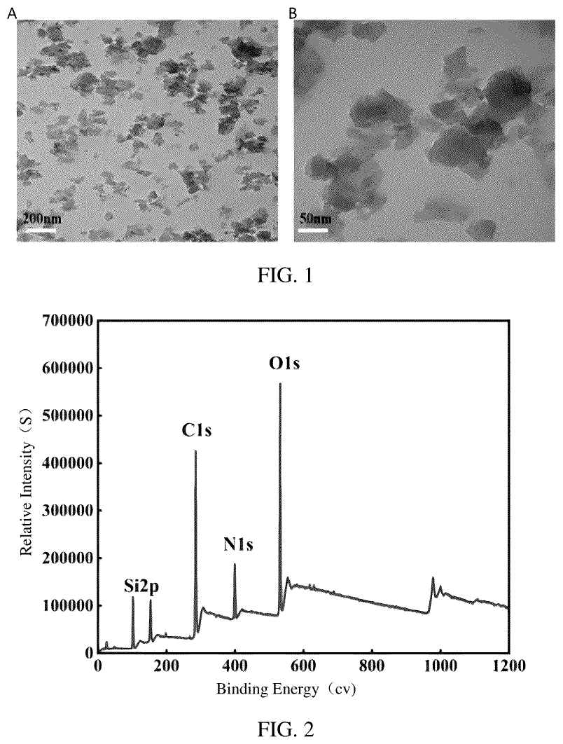

The morphology of the Si-CQDs@DMIPs material was characterized using a scanning electron microscope (SEM), with the instrument model being Tecnai G2 F20. The SEM image is shown in FIG. 1. The results indicated that the particle size of the prepared Si-CQDs@DMIPs material was 25 nm.

### (2) Preparation Results and Fluorescence Intensity Evaluation of Silane-Functionalized Quantum Dots:

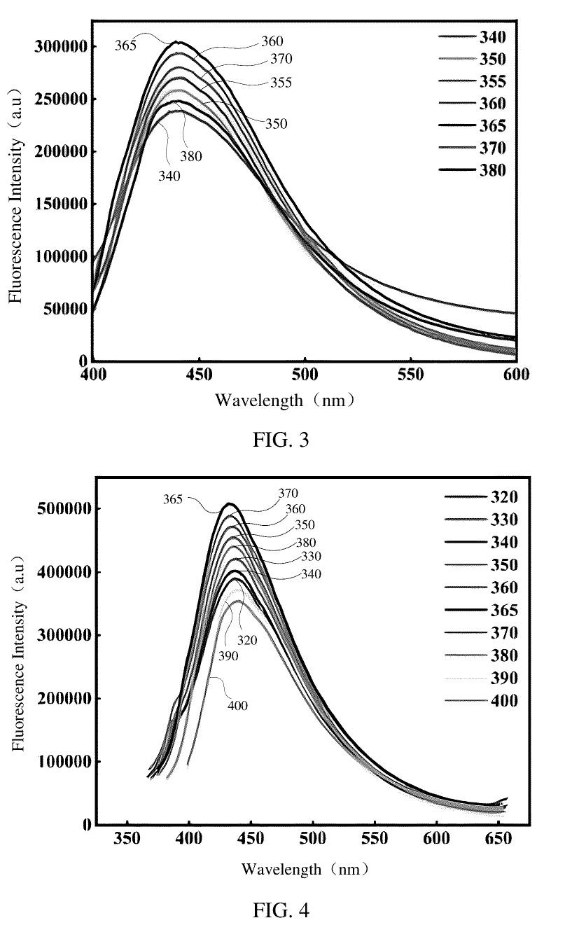

\textcircled{1} The chemical composition of the prepared silane-functionalized quantum dots was analyzed using XPS. The test results are shown in FIG. 2. The four peaks on the XPS energy spectrum represent Si2p (102.08 eV), C1s (285.08 eV), N1s (399.08 eV), and O1s (532.08 eV), respectively, which are consistent with the chemical composition of the prepared quantum dots, indicating the successful synthesis of silane-functionalized CQDs.
\textcircled{2} The fluorescence intensity of the silane-functionalized quantum dots was measured using a fluorescence spectrophotometer. As shown in FIG. 3, the silane-functionalized CQDs exhibit a significant emission wavelength in the range of 400 nm to 600 nm and can produce dazzling blue light under a UV lamp. According to FIG. 3, the height of their emission spectrum is closely related to the excitation wavelength. As the excitation wavelength increases from 340 nm to 365 nm, the fluorescence intensity also increases. The fluorescence intensity reaches its maximum at an excitation wavelength of 365 nm. However, as the excitation wavelength continues to increase, the fluorescence intensity gradually decreases.

### (3) Fluorescence Performance of Si-CQDs@DMIPs and Quantitative Determination of Bisphenol A:

As can be seen from FIG. 4, the emission wavelength of Si-CQDs@DMIPs is dependent on the excitation wavelength. As the excitation wavelength increases, the fluorescence intensity gradually increases, and the height of the emission wavelength also changes accordingly. When excited at 365 nm, the fluorescence spectrum shows the highest fluorescence intensity. However, as the excitation wavelength continues to increase, the fluorescence intensity gradually decreases.

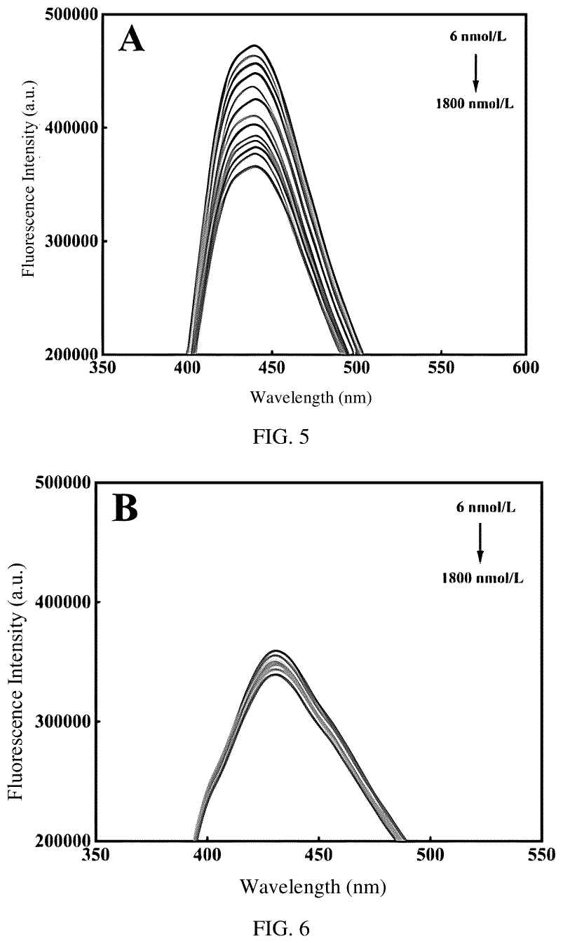

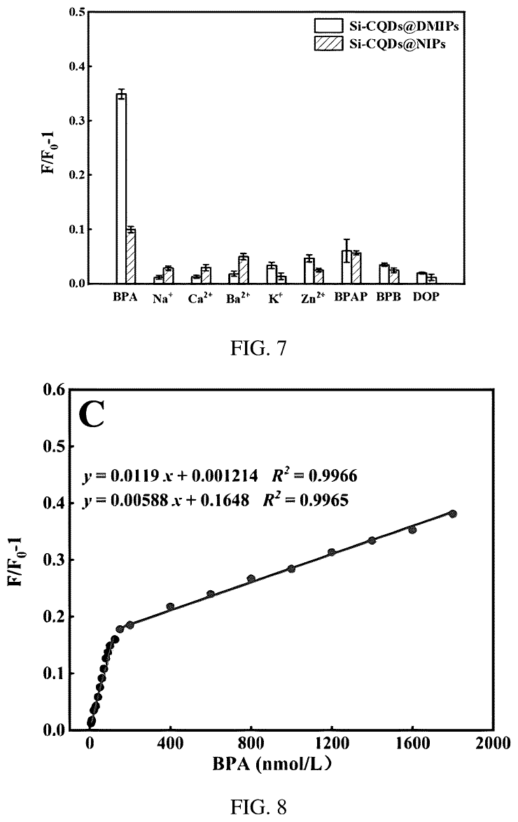

As shown in FIG. 5, the fluorescence intensity of Si-CQDs@DMIPs decreases with increasing BPA concentration. By calculating the changes in fluorescence intensity and BPA concentration, a linear relationship between the change in fluorescence intensity of Si-CQDs@DMIPs and BPA concentration is obtained. As shown in FIG. 8, when the BPA concentration linearly increases from 6-1800 nmol/L, the change in fluorescence intensity of Si-CQDs@DMIPs shows a two-segment linear relationship with BPA concentration. When the BPA concentration is in the range of 6-150 nmol/L, y=0.0119x+0.001214, R²=0.9966. When the BPA concentration is in the range of 150 nmol/L-1800 nmol/L, y=0.00588x+0.1648, R²=0.9965, where y represents the vertical coordinate F/F₀⁻¹, and x represents the added C_{BPA} concentration. The limit of detection (LOD) can be calculated as 0.65 nmol/L based on 3σ/k (where σ is the standard deviation of the blank sample and k is the slope of the standard curve).

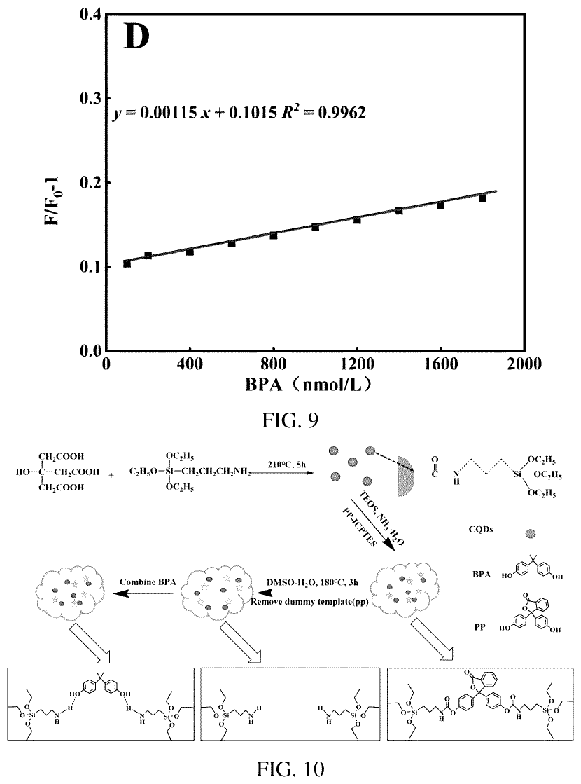

For ease of comparison, FIG. 6 shows the curve of the fluorescence intensity of Si-CQDs@NIPs changing with BPA concentration, and FIG. 9 shows the linear range diagram for BPA detection by Si-CQDs@NIPs (100-1800 nmol/L) further calculated based on FIG. 6, with a linear relationship of y=0.00115x+0.1015, R²=0.9962, LOD=1.77 nmol/L, where y represents the vertical coordinate F/F₀⁻¹, and x represents the added C_{BPA} concentration. Compared with FIG. 5, although both Si-CQDs@DMIPs and Si-CQDs@NIPs show fluorescence quenching responses to BPA, the detection sensitivity and linear range of Si-CQDs@DMIPs for BPA are significantly higher than those of Si-CQDs@NIPs, further proving the successful construction of imprinted recognition sites, which exhibit obvious imprinted selectivity.

### (4) Anti-Interference Selectivity Test of Si-CQDs@DMIPs:

The selectivity experiment evaluates the anti-interference ability of Si-CQDs@DMIPs in a complex matrix environment, using Si-CQDs@NIPs as a control. The concentrations of both Si-CQDs@DMIPs and Si-CQDs@NIPs in the system are 0.2 mg/L. The selectivity of Si-CQDs@DMIPs is tested by measuring the degree of interference from other components in the mixture on the specific analyte in the complex mixture. Metal ions (including Na⁺, Ca²⁺, Ba²⁺, K⁺, Zn²⁺), BPA structural analogs (BPB, BPAP), and dioctyl phthalate (DOP) are selected as potential interferents in the matrix environment, each at a concentration of 0.05 g/L. The results are shown in FIG. 7, where the selectivity of the prepared Si-CQDs@DMIPs for BPA detection is assessed through the fluorescence change rate (F/F₀⁻¹). The imprinting factor, calculated from the ratio of F/F₀⁻¹ for Si-CQDs@DMIPs to that for Si-CQDs@NIPs, is 1.5. This indicates that when the concentration of Si-CQDs@DMIPs is 0.2 mg/L and the concentration of other interfering substances is 0.05 g/L, Si-CQDs@DMIPs exhibits high selectivity for BPA, and the interfering substances have minimal impact on the detection results. Therefore, this method can be used for the detection of BPA in Baijiu stored in plastic barrels and for assessing potential adverse effects of plastic barrels on Baijiu quality. (5) Accuracy verification of BPA testing using Si-CQDs@DMIPs

A certain amount of Si-CQDs@DMIPs and a specific volume of Baijiu solution are taken to achieve a final concentration of 0.2 mg/L for Si-CQDs@DMIPs. After thorough reaction, the fluorescence change rate is tested. To verify the accuracy of the method, a standard addition recovery experiment is conducted (with BPA concentrations of 50, 200, 500, and 1000 nM). The recovery rates for BPA obtained using this method range from 81.5% to 93.8%, with relative standard deviations (RSDs, n=5) ranging from 3.0% to 5.8%. This indicates that the method using Si-CQDs@DMIPs for detecting BPA in Baijiu has high accuracy.

### (6) Application of Si-CQDs@DMIPs for the Detection of Baijiu Samples

Si-CQDs@DMIPs were used for the detection of bisphenol A in 10 different commercially available Baijiu samples packaged in plastic barrels. The final concentration of Si-CQDs@DMIPs in the system was 0.2 mg/L. The fluorescence change rate in the system was measured, and the bisphenol A content was calculated. The final results showed that bisphenol A was not detected in 9 out of the 10 Baijiu samples, while it was detected in 1 sample.

In the specification and claims of the present disclosure, certain terminology will be used to refer to specific products. Those skilled in the art should understand that manufacturers may use different names to refer to the same components. This document is not intended to distinguish between components that have the same function but different names. Throughout the subsequent specification and scope of the patent application, the terms “comprising”, “having”, and “including” are open-ended terms and should therefore be interpreted to mean “including but not limited to . . . ”.

The above descriptions are preferred embodiments of the present disclosure, and it should be noted that, for a person of ordinary skill in the art, several improvements and modifications can be made without departing from the principle of the present disclosure, and these improvements and modifications are also considered to be within the protection scope of the present disclosure.

What is claimed is:

1. A preparation method of a nanogel fluorescent sensor, comprising the following steps:
(1) preparing carbon quantum dots:
(1-1) performing a reaction in a sealed environment using anhydrous citric acid as a carbon source and 3-aminopropyltriethoxysilane as a dispersant;
(1-2) after the reaction is finished, centrifuging is performed to remove unreacted solids, obtaining silane-functionalized quantum dots, dispersing the silane-functionalized quantum dots in anhydrous ethanol, and storing at 4° C. for later use;
(2) preparing an alternative template-monomer covalent complex:
(2-1) dissolving bisphenol A, acting as an alternative for template molecules phenolphthalein and 1,1,1-tris (4-hydroxyphenyl) ethane, in N,N-dimethylformamide, then adding a functional monomer isocyanatopropyltriethoxysilane, and subsequently performing a sealed reaction at 60-90° C. for 24-72 h;
(2-2) after the reaction is finished, removing N,N-dimethylformamide from the system to obtain a bisphenol A alternative template-monomer covalent complex, and storing the bisphenol A alternative template-monomer covalent complex under high-purity nitrogen sealing for later use;
(3) preparing an alternative template molecularly imprinted nanogel fluorescent sensor using carbon quantum dots as a fluorescent unit:
(3-1) dissolving the bisphenol A alternative template-monomer covalent complex in anhydrous ethanol to form a pre-reaction solution;
(3-2) dropwise adding a silane-functionalized quantum dot ethanol solution into the pre-reaction solution under mechanical stirring, dropwise adding tetraethyl orthosilicate and an ammonia solution in sequence, and stirring the mixture at room temperature for 2-4 h;
(3-3) after the reaction is completed, performing solid-liquid separation on the mixture to obtain a gel material; and
(3-4) adding the gel material into a dimethyl sulfoxide aqueous solution, maintaining the mixture at 160-200° C. for 2-4 h to remove the bisphenol A alternative template molecule, separating out a solid, then cleaning the solid, and carrying out vacuum drying to obtain the alternative template molecularly imprinted nanogel fluorescent sensor.

2. The preparation method according to claim 1, wherein in step (1-1), anhydrous citric acid is fully dissolved in 3-aminopropyltriethoxysilane with the assistance of ultrasound, where a molar ratio of citric acid to 3-aminopropyltriethoxysilane is 1:14-16; and/or, performing the reaction in the sealed environment is maintained at 180-240° C. for 3-6 h, where performing the reaction in the sealed environment is maintained at 210±5° C. for 6 h; and performing the reaction in the sealed environment is carried out in either a graphene reaction kettle or a Teflon-lined autoclave.

3. The preparation method according to claim 1, wherein in step (1-2), the silane-functionalized quantum dots are dispersed in anhydrous ethanol according to a volume ratio of 1:1.

4. The preparation method according to claim 1, wherein in step (2-1), a molar ratio of the bisphenol A alternative template molecule to the functional monomer isocyanatopropyltriethoxysilane is 1:1.5-4, where the molar ratio of the bisphenol A alternative template molecule to the functional monomer isocyanatopropyltriethoxysilane is 1:2-3; and/or, the sealed reaction is conducted in a high-pressure glass reaction tube, where the sealed reaction is conducted at 80±5° C. for 48-50 h; and/or, N,N-dimethylformamide is a super-dry solvent.

5. The preparation method according to claim 1, wherein in step (3-1), the bisphenol A alternative template-monomer covalent complex is dissolved in anhydrous ethanol by ultrasound, where a concentration of the bisphenol A alternative template-monomer covalent complex is 0.01-0.1 mol/L.

6. The preparation method according to claim 1, wherein in step (3-2), a mass ratio of the bisphenol A alternative template-monomer covalent complex to the silane-functionalized quantum dots in the pre-reaction solution is 1:5-20, where the mass ratio of the bisphenol A alternative template-monomer covalent complex to the silane-functionalized quantum dots in the pre-reaction solution is 1:10; and/or, a mechanical stirring speed is 200-400 rpm; and/or, a molar ratio of tetraethyl orthosilicate to the bisphenol A alternative template-monomer covalent complex is 7-10:1, where the molar ratio of tetraethyl orthosilicate to the bisphenol A alternative template-monomer covalent complex is 8:1; and/or, a ratio of the ammonia solution to the bisphenol A alternative template-monomer covalent complex is 1-2:1 mL/mmol, where the ratio of the ammonia solution to the bisphenol A alternative template-monomer covalent complex is 1:1 mL/mmol.

7. The preparation method according to claim 1, wherein in step (3-3), the solid-liquid separation method is centrifugation, where control parameters of the centrifugation are: a rotation speed is 8000-12000 rpm, and a centrifugation time is 10-15 min.

8. The preparation method according to claim 1, wherein in step (3-4), a volume ratio of dimethyl sulfoxide to water is 5-10:1.

9. A bisphenol A detection kit, comprising the nanogel fluorescent sensor obtained by the preparation method according to claim 1.

10. A method for detecting bisphenol A in plastic barrel-packed Baijiu, wherein the kit according to claim 9 is adopted, comprises the following steps:
mixing a nanogel fluorescent sensor in the kit with Baijiu, and measuring a fluorescence change rate in a system; and
calculating a content of bisphenol A in Baijiu based on the fluorescence change rate.

11. The method according to claim 10, wherein a final concentration of the nanogel fluorescent sensor in the system is 0.1-0.5 mg/L.
